(19) United States
(12) Patent Application Publication
(10) Pub. No.: US 2006/0052287 A1
(43) Pub. Date: Mar. 9, 2006

(54) Polyethylene glycol - peptide copper complexes and compositions and methods related thereto

(75) Inventor: Leonard M. Patt, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: ProCyte Corporation, Redmond, WA (US)

(21) Appl. No.: 11/204,772

(22) Filed: Aug. 16, 2005

Related U.S. Application Data
(60) Provisional application No. 60/602,715, filed on Aug. 18, 2004.

(51) Int. Cl. A61K 38/16

(52) U.S. Cl. 514/6

(57) ABSTRACT

This invention relates to compositions comprising polyethylene glycol molecules coupled to peptide copper complexes, and, additionally, to such compositions formulated for use as pharmaceutical and cosmetic products, as well as to medical devices that comprise such compositions.

### CROSS-REFERENCE TO RELATED APPLICATION

This application claims the benefit under 35 U.S.C. § 119(e) of U.S. Provisional Patent Application No. 60/602,715 filed Aug. 18, 2004, which provisional application is incorporated herein by reference in its entirety.

### BACKGROUND OF THE INVENTION

1. Field of the Invention

The present invention relates to compositions comprising polyethylene glycol (PEG) molecules coupled to peptide copper complexes, and, additionally, to such compositions formulated for use as pharmaceutical and cosmetic products, as well as to medical devices that comprise such compositions.

2. Description of the Related Art

Copper is known to have many beneficial biological applications, including, as a few examples, stimulating the accumulation of collagen and elastin in wounds (see, e.g., Maquart et al., FEBS Lett. 238(2):343-346 (1988); Maquart et al., J. Clin. Invest. 92:2368-2376 (1993); and Wegrowski et al., Life Sci. 51(13):1049-1056 (1992)) and in intact skin (see, e.g., Abdulghani et al, Disease Management and Clinical Outcomes 1(4):136-141 (1998)), modulating the activity of matrix metalloproteases (see, e.g., Simeon et al., J. Invest. Dermatol. 112:957-964 (1999); and Canapp et al., Veterinary Surgery 32(6):515-523 (2003)), increasing angiogenesis (see, e.g., Raju et al., JNCI 69(5):1183-1188 (1982); Hu et al., J. Cell. Biochem. 69:326-335(1998); and Lane et al., J. Cell. Biol. 125(4):929-943 (1994)), increasing the rate of wound healing (see, e.g., Branca et al., Clinical Research 40: 75a (1992)), inhibiting 5-alpha reductase, a key enzyme in regulating hair growth (see, e.g., Sugimoto et al., J. Invest. Dermatol. 104(5):775-778 (1995)), and stimulating hair growth (see, e.g., Perez-Meza et al., Inter. J. Cosmetic Surgery 6(1):80-84 (1998); Trachy et al., Phototrichogram Analysis of Hair Follicle Stimulation: A Pilot Clinical Study with a Peptide Copper Complex, in Dermatologic Research Techniques 217-226 (Maibach ed., 1996); Trachy et al., Quantitative Assessment of Peptide-Copper Complex-Induced Hair Follicle Stimulation Using the Fuzzy Rat, in Dermatologic Research Techniques 227-239 (Maibach ed., 1996); and Trachy et al., Ann. N.Y. Acad. Sci. 642:468-469 (1991)).

However, copper salts are generally ineffective, or even inhibitory, for such applications. The copper ion must be delivered in a biologically acceptable form. As an example, when copper is complexed with a biologically acceptable carrier molecule, such as a peptide or protein, it may then be effectively delivered to cells and tissues.

Specifically, peptide copper complexes, and compositions comprising the same, may be effective in this regard. Peptide copper complexes that are useful for wound healing and skin health are disclosed in U.S. Pat. Nos. 4,760,051; 4,665,054; 4,877,770; 5,135,913 and 5,348,943. Peptide copper complexes, beneficial for stimulating hair growth and preventing hair loss, are disclosed in U.S. Pat. Nos. 5,177,061; 5,214,032; 5,120,831; 5,550,183 and 5,538,945. Another beneficial application of peptide copper complexes is the prevention and healing of gastric ulcers, as disclosed in U.S. Pat. Nos. 5,145,838; 4,767,753 and 5,023,237. Yet other utilities of such complexes are the healing of bone, as disclosed in U.S. Pat. No. 5,059,588, and the coupling of the copper peptide complex of glycyl-L-histidyl-L-lysine (GHK) to solid implantable polymers of lactic acid and the like, as. disclosed in U.S. Pat. No. 5,386,012.

While peptide copper complexes have shown utility in areas such as wound healing, skin health, and hair growth, there remains a need for more efficient ways to deliver peptide copper complexes to areas of the body which would benefit from their activity. Some of the above U.S. patents, namely U.S. Pat. Nos. 5,177,061, 5,059,588, and 5,386,012, disclose peptide copper complexes which are coupled to various molecular groups, such as fatty acids and the like, to alter certain properties of the peptide copper complexes with the aim of increasing their solubility in, for example, organic solvents, increasing their skin permeability, or decreasing their susceptibility to proteolytic cleavage.

As disclosed by Kawase et al., the tripeptide glycyl-L-histidyl-L-lysine (GHK) has been coupled to polyamidoamine dendrimers to function as a substrate for the growth of hepatoma cells in culture (see, Kawase et al, J. Bioscience and Bioengineering 88(4):433-437 (1999)). The disclosed polyamidoamine dendrimers are spherical highly branched polymers used as a substrate for artificial liver experimentation. It was found that addition of GHK aided in the immobilization of the hepatoma cells on the substrate. As disclosed, the GHK was complexed with either copper or zinc. In a similar manner, GHK has been coupled to poly(vinylalcohol)-quarternized stilbazole to serve as an attachment site for hepatoma cells (see, Kawase et al., Biological and Pharmaceutical Bulletin 22(9):999-1001 (1999)).

Zheng et al. describe the immobilization of GHK within a photoreversible hydrogel composed of modified polyethylene glycol. The GHK was either entrapped within the gel matrix or covalently bound (see, Zheng et al., “Design of Photoreversible Hydrogels Aimed at Biomedical Applications,” Abstracts of Papers, 221st ACS National Meeting, San Diego, Calif., Apr. 1-5, 2001 (2001)). It was suggested that such a hydrogel would show utility in wound healing or drug delivery.

In addition to more efficient ways to deliver peptide copper complexes to areas of the body that would benefit from their activity, there also remains a need for ways to increase the length of time a peptide copper complex remains at the site of application. The present invention fulfills these needs and provides further related advantages.

### BRIEF SUMMARY OF THE INVENTION

In brief, the present invention is directed to compositions comprising polyethylene glycol—peptide copper complexes having utility as pharmaceutical and cosmetic products. It has been found that by coupling a polyethylene glycol (PEG) molecule to a peptide copper complex, the increased molecular size increases the length of time of the PEG-peptide copper complex at, for example, an injection site or site of topical application. In addition, the increased molecular size and type of chemical bond coupling the PEG molecule to the peptide copper complex results in a measured release of the peptide copper complex or copper at a site, such as a hard or soft tissue implant site.

As previously noted, peptide copper complexes, and compositions comprising the same, have beneficial utility for, as some examples, skin health and appearance; wound healing; hair, bone and tissue growth; and hair loss prevention. Accordingly, the present invention, in another embodiment, is directed to a cosmetic or pharmaceutical composition suitable for use as a pharmaceutical or cosmetic product, such as a topical formulation suitable for wound care. In another embodiment, disclosed is a medical device, such as an implantable medical device or a wound dressing that comprises a composition of the present invention.

Generally, the composition of the present invention comprises a peptide copper complex coupled to a polyethylene glycol molecule. In more specific embodiments, the peptide copper complex has the formula:
[R₁—R₂—R₃]:copper(II)
wherein R₁ is an amino acid or an amino acid derivative, R₂ is an amino acid or an amino acid derivative, and R₃ is at least one amino acid or amino acid derivative or a chemical moiety.

In yet further, more specific embodiments, (1) R₂is L-histidyl and R₁ and R₃ are both naturally occurring amino acids, R₁ is a naturally occurring amino acid and R₃ is L-lysine, R₁ is glycyl and R₃ is L-lysine, or R₁ is L-alanyl and R₃ is L-lysine, (2) R₂ is L-histidyl(R), wherein R is 3-methyl, 5 methyl, 3-ethyl or 5-ethyl, and R₁ and R₃ are both naturally occurring amino acids, or (3) R₂ is a L-arginyl and R₁ and R₃ are both naturally occurring amino acids.

These and other aspects of the present invention will be evident upon reference to the following detailed description of the invention.

### DETAILED DESCRIPTION OF THE INVENTION

As noted above, in one embodiment of the present invention, disclosed is a composition comprising a peptide copper complex coupled to a polyethylene glycol molecule.

In more specific embodiments, the peptide copper complex is glycyl-L-histidyl-L-lysine:copper(II) (“GHK—Cu”), L-alanyl-L-histidyl-L-lysine:copper(II) (“AHK—Cu”), L-valyl-L-histidyl-L-lysine:copper(II) (“VHK—Cu”), L-leucyl-L-histidyl-L-lysine:copper(II) (“LHK—Cu”), L-isoleucyl-L-histidyl-L-lysine:copper(II) (“IHK—Cu”), L-phenylalanyl-L-histidyl-L-lysine:copper(II) (“FHK—Cu”), L-prolyl-L-histidyl-L-lysine:copper(II) (“PHK—Cu”), L-seryl-L-histidyl-L-lysine:copper(II) (“SHK—Cu”), or L-threonyl-L-histidyl-L-lysine:copper(II) (“THK—Cu”).

As used herein, the expression “peptide copper complex” generally refers to a coordination compound comprising a peptide molecule and a copper(II) ion non-covalently complexed with the peptide. As is well understood in the art, copper (II) designates a copper ion having a valence of 2 (i.e., Cu⁺²). The peptide molecule is a chain of two or more amino acids or amino acid derivatives covalently bonded together. Generally, an amino acid consists of an amino group, a carboxyl group, a hydrogen atom, and an amino acid side-chain moiety—all bonded, in the case of an alpha-amino acid, to a single carbon atom that is referred to as an alpha-carbon. However, the amino acids of the present invention may be provided by amino acids other than alpha-amino acids. For example, the amino acids may be beta- or gamma-amino acids, such as those shown below. 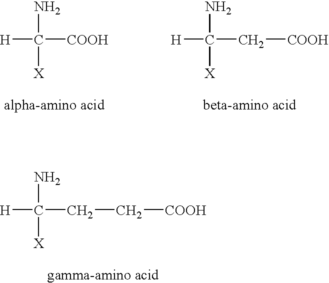
where X is the amino acid side-chain moiety bonded, along with the amino group and hydrogen, to an alpha-, beta-, or gamma-carbon atom.

The amino acids of the present invention include, but are not limited to, naturally occurring alpha-amino acids. Naturally occurring amino acids are those from which the amino acids of naturally occurring proteins are derived. The respective amino acid side-chain moieties of these amino acids, are shown below in Table 1. The naturally occurring amino acids are all in the L configuration, referring to the optical orientation of the alpha carbon or other carbon atom bearing the amino acid side-chain moiety. However, a peptide molecule of the present invention may also comprise amino acids that are in the D optical configuration, or a mixture of D and L amino acids.

TABLE 1
Naturally Occurring Amino Acid Side-Chain Moieties

| Amino Acid Side-Chain Moiety | Amino Acid |
|---|---|
| —H | Glycine |
| —CH₃ | Alanine |
| —CH(CH₃)₂ | Valine |
| —CH₂CH(CH₃)₂ | Leucine |
| —CH(CH₃)CH₂CH₃ | Isoleucine |
| —(CH₂)₄NH₃⁺ | Lysine |
| —(CH₂)₃NHC(NH₂)NH₂⁺ | Arginine |
| 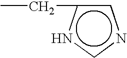 | Histidine |
| —CH₂COO— | Aspartic Acid |
| —CH₂CH₂COO— | Glutamic Acid |
| —CH₂CONH₂ | Asparagine |
| —CH₂CH₂CONH₂ | Glutamine |
| 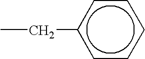 | Phenylalanine |
| 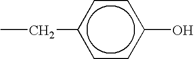 | Tyrosine |
| 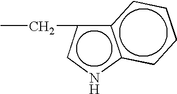 | Tryptophan |
| —CH₂SH | Cysteine |
| —CH₂CH₂SCH₃ | Methionine |
| —CH₂OH | Serine |
| —CH(OH)CH₃ | Threonine |
| 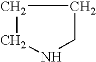 | Proline |

Other naturally occurring amino acids include hydroxyproline and gamma-carboxyglutamate.

Representative amino acid derivatives include those set forth in Table 2 below.

TABLE 2
Representative Amino Acid Derivatives

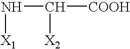

Where X₂ = H or the following moieties:

—(CH₂)ₙCH₃ where n = 1-20
—(CH₂)ₙCH(CH₃)(CH₂)ₘCH₃
where n, m = 0-20 (when n = 0, m ≠ 0 or 1 and when n = 1, m ≠ 0)
—(CH₂)ₙNH₂ where n = 1-20 (n ≠ 4)
—(CH₂)ₙCONH₂ where n = 3-20
—(CH₂)ₙCOOH where n = 3-20

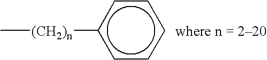

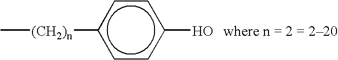

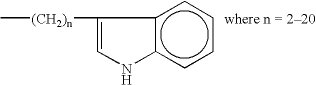

—(CH₂)ₙSH where n = 2-20
—(CH₂)ₙS(CH₂)ₘCH₃ where n, m = 1-20 (when n = 2, m ≠ 0)
—(CH₂)ₙCH₂OH where n = 1-20
—(CH₂)ₙCH(CH₃)OH where n = 1-20

And where X₁ = H or the following moieties:
—(CH₂)ₙCH₃ where n = 0-20
—(CH₂)ₙCH(CH₃)(CH₂)ₘCH₃ where n, m = 0-20

For example, histidine derivatives of this invention include compounds having the structure: 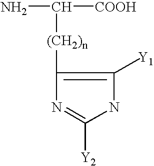
where n=1-20, and Y₁ and Y₂ are independently selected from alkyl moieties containing from 1-12 carbon atoms or an aryl moiety containing from 6-12 carbon atoms. In certain embodiments, n is 1, Y₂ is methyl or ethyl, and Y₁ is H (i.e., 3-methyl histidine or 3-ethyl histidine, respectively) or Y₂ is H and Y₁ is methyl or ethyl (i.e., 5-methyl histidine or 5-ethyl histidine, respectively).

Similarly, arginine derivatives of this invention include compounds having the structure: 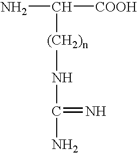
where n=1-20 (excluding n=3).

As used herein, “alkyl” means a straight chain or branched, cyclic or noncyclic, substituted or unsubstituted, saturated or unsaturated aliphatic hydrocarbon containing from 1 to 18 carbon atoms. Representative saturated straight chain alkyls include methyl, ethyl, n-propyl and the like; while saturated branched alkyls include isopropyl, sec-butyl, isobutyl, tert-butyl, isopentyl, and the like. Representative, saturated cyclic alkyls include cyclopropyl, cyclobutyl, cyclopentyl, —CH₂cyclohexyl, and the like; while unsaturated cyclic alkyls include cyclopentenyl, cyclohexenyl, and the like. Unsaturated alkyls contain at least one double or triple bond between adjacent carbon atoms (referred to as an “alkenyl” or “alkynyl”, respectively). Representative alkenyls include ethylenyl, 1-butenyl, isobutylenyl, 2-methyl-2-butenyl, and the like; while representative alkynyls include acetylenyl, 2-butynyl, 3-methyl-1-butynyl, and the like.

Also, as used herein, “aryl” means an aromatic carbocyclic moiety such as phenyl or naphthyl, and may be substituted or unsubstituted. “Arylalkyl,” as used herein, means an alkyl having at least one alkyl hydrogen atom replaced with a substituted or unsubstituted aryl moiety, such as benzyl (i.e., —CH₂phenyl, —(CH₂)₂phenyl, —(CH₂)₃phenyl, —CH(phenyl)₂, and the like).

A peptide copper complex of the present invention may have the formula [R₁—R₂—R₃]:copper(II) where R₃ is at least one amino acid or amino acid derivative, as defined above, bonded to R₂ by a peptide bond. Where R₃ is a single amino acid or amino acid derivative, then the peptide of the peptide copper complex is generally classified as a tripeptide. As another example of a peptide copper complex of the present invention having the formula [R₁—R₂—R₃]:copper(II), R₃ is a chemical moiety bonded to the R₂ moiety by an amide bond. The expression “chemical moiety,” as used herein and with reference to R₃, includes any chemical moiety having an amino group capable of forming an amide bond with the carboxyl terminus of R₂ (e.g., the carboxyl terminus of histidine, arginine, or derivatives thereof). Further examples of suitable chemical moieties having amino groups capable of forming an amide linkage with the carboxyl terminus of R₂ include polyamines, such as spermine and sperimidine.

It should be understood that R₃ may include more than one chemical moiety. For example, additional amino acids, chains of amino acids, or amino acid derivatives may be bonded to the above-described peptide copper complexes comprising tripeptides to yield peptide copper complexes comprising peptides having four or more amino acids and/or amino acid derivatives. For purposes of illustration, Table 3, shown below, presents various representative examples of peptide copper complexes of the present invention.

TABLE 3
Representative Peptide-Copper Complexes
Examples of [R₁—R₂]:copper(II)

| | |
|---|---|
| glycyl-histidine:copper | alanyl-histidine:copper |
| glycyl-(3-methyl)histidine:copper | alanyl-(3-methyl)histidine:copper |
| glycyl-(5-methyl)histidine:copper | alanyl-(5-methyl)histidine:copper |
| glycyl-arginine:copper | alanyl-arginine:copper |
| (N-methyl)glycine-histidine:copper | (N-methyl)glycine-arginine:copper |

Examples of [R₁—R₂—R₃]:copper(II)
where R₃ is Chemical Moiety Linked by Amide Bond

| | |
|---|---|
| glycyl-histidyl-NH₂:copper | glycyl-arginyl-NH₂:copper |
| glycyl-(3-methyl)histidyl-NH₂:copper | alanyl-(3-methyl)histidyl-NH₂:copper |
| glycyl-arginyl-NH₂:copper | alanyl-arginyl-NH₂:copper |
| (N-methyl)glycine-histidyl-NH₂:copper | (N-methyl)glycine-arginyl-NH₂:copper |
| glycyl-histidyl-NHoctyl:copper | glycyl-arginyl-NHoctyl:copper |

Examples of [R₁—R₂—R₃]:copper(II)
where R₃ is Amino Acid or Amino Acid Derivative
Linked by Peptide Bond

| | |
|---|---|
| glycyl-histidyl-lysine:copper | glycyl-arginyl-lysine:copper |
| glycyl-(3-methyl)histidyl-lysine:copper | glycyl-(5-methyl)histidyl-lysine:copper |
| alanyl-histidyl-lysine:copper | alanyl-arginyl-lysine:copper |
| alanyl-(3-methyl)histidyl-lysine:copper | alanyl-(5-methyl)histidyl-lysine:copper |
| glycyl-histidyl-phenylalanine:copper | glycyl-arginyl-phenylalanine:copper |
| glycyl-(3-methyl)histidyl- | glycyl-(5-methyl)histidyl- |
| phenylalanine:copper | phenylalanine:copper |
| alanyl-histidyl-phenylalanine:copper | alanyl-arginyl-phenylalanine:copper |
| alanyl-(3-methyl)histidyl- | alanyl-(5-methyl)histidyl- |
| phenylalanine:copper | phenylalanine:copper |
| glycyl-histidyl-lysyl-phenylalanyl- | glycyl-arginyl-lysyl-phenylalanyl- |
| phenylalanyl:copper | phenylalanyl:copper |
| glycyl-(3-methyl)histidyl-lysyl- | glycyl-(5-methyl)histidyl-lysyl- |
| phenylalanyl-phenylalanyl:copper | phenylalanyl-phenylalanyl:copper |
| (N-methyl)glycyl-histidyl-lysine:copper | (N-methyl)glycyl-arginyl-lysine:copper |
| valyl-histidyl-lysine:copper | glycyl-histidyl-lysyl-prolyl-phenylalanyl-proline:copper |
| prolyl-histidyl-lysine:copper | Leucyl-histidyl-lysine:copper |
| glycyl-D-histidyl-L-lysine:copper | |
| seryl-histidyl-lysine:copper | |

Further examples of peptide copper complexes encompassed by embodiments of the present invention are disclosed in U.S. Pat. Nos. 4,665,054; 4,760,051; 4,767,753; 4,810,693; 4,877,770; 5,023,237; 5,059,588; 5,118,665; 5,120,831; 5,164,367; 5,177,061; 5,214,032; 5,538,945; 5,550,183; and 6,017,888, all of which are incorporated herein by reference in their entirety.

Yet further examples of the peptide copper complexes encompassed by the present invention, include, but are not limited to, those disclosed and described in the published PCT application having the International Publication Number WO 94/03482, which is incorporated herein by reference in its entirety.

The synthesis of the above-disclosed peptide copper complexes is described in detail in the above-referenced patents. For example, the peptides of the peptide copper complexes disclosed herein may be synthesized by either solution or solid phase techniques known to one skilled in the art of peptide synthesis. The general procedure involves the stepwise addition of protected amino acids to build up the desired peptide sequence. The resulting peptide may then be complexed to copper (at the desired molar ratio of peptide to copper) by dissolving the peptide in water, followed by the addition of copper chloride or other suitable copper salt and adjusting the pH to greater than 4.0.

In more specific embodiments of the compositions of the present invention, the molar ratio of peptide to copper in the peptide copper complexes ranges from 1:1 to 3:1. In other more specific embodiments, the peptide copper complex is present at a concentration, by weight of the composition, ranging from about 0.05% to about 25%; from about 0.05% to about 2%; and from about 0.1% to about 0.5%.

In other embodiments of the present invention, the peptide portion of the PEG-peptide copper complex may be of natural origin. In such embodiments, the peptide is formed by the hydrolysis of naturally occurring proteins, polypeptides, or larger peptides of either plant, microbial, or animal origin. Hydrolysis may be by enzymatic treatment or by acid or base hydrolysis. The copper complex of this type of peptide copper complex is formed by addition of a suitable copper salt to the aqueous solution of the peptide. Alternatively, the peptide copper complex may be formed during the manufacturing of a formulation by separate additions of the peptide and copper salt in a suitable solvent.

As noted above, the present invention is directed to compositions comprising a peptide copper complex coupled to a polyethylene glycol molecule.

Polyethylene glycol (PEG) molecules are polymers comprising repeating units of ethylene glycol, and may be either branched or linear. A typical linear structure is shown below. As further shown, the terminal hydroxyl, HO—, groups may be further modified with, for example, methyl or ethyl groups, amino groups, reactive groups, and the like to further modify the solubility and reactive properties of the PEG molecule.
HO—(CH₂CH₂O)ₙ—CH₂—CH₂—OH PEG
CH₃O—(CH₂CH₂O)ₙ—CH₂—CH₂—OH Methyl-PEG
Branched PEG molecules comprise two (bi), four (tetra), eight (octa), or more polyethylene glycol side (secondary) chains extending from the main (primary) polyethylene glycol backbone.

The polyethylene glycol molecules of the present invention may have a wide range of molecular weights. For example, in certain specific embodiments, a representative polyethylene glycol molecule may have a molecular weight of about 5,000 daltons, about 20,000, about 30,000 daltons, or about 40,000 daltons.

There are a number of methods for coupling peptides to polyethylene glycol molecules, referred to as pegylation (PEGylation), which are well known to one skilled in the art. For example, human growth hormone was coupled to a 5,000 molecular weight PEG molecule by the method set forth in Clark et al., J. Biol. Chem. 271: 21969-21977 (1996). Namely, PEG (5,000 MW) N-hydroxysuccinimide (PEG-NHS) was reacted with human growth hormone in 0.05M sodium borate buffer at pH 8.5 for 30-60 minutes at room temperature. The molar ratio of PEG-NHS to lysine amino groups was 3 to 1. The reaction was terminated by the addition of 0.05M Tris buffer, pH 7.5. In alternate method, disclosed in Abuchowski et al., J. Biol. Chem. 252: 3578-3581 (1977), a PEG molecule was initially directly activated with cyanuric chloride and the activated PEG was reacted with albumin at pH 9.2 in sodium tetraborate buffer. The reaction was terminated after 1 hour by decreasing the pH to 7.3 with phosphate buffer.

Representative examples of functional molecules that can provide stable linkages to peptides are succinimidyl esters, aldehydes, benzotriazole, and thioesters. These reactive groups may be coupled to the PEG molecule or modified PEG molecule, and then coupled with, for example, the lysine amino group of a peptide.

In this way, a representative PEG-peptide copper complex of the present invention may be prepared by coupling a peptide, as described above, to a PEG molecule to form a pegylated peptide. The resulting PEG-peptide, for example, PEG-GHK, is then complexed with copper to form the PEG-GHK:copper complex. In a similar manner, PEG-AHK, PEG-VHK, and the like can be prepared and complexed with copper.

In embodiments of the present invention, the PEG molecule may be coupled to the peptide portion of a peptide copper complex using a linkage group which is slowly hydrolyzed, or broken down, in a biological environment. In this way, a slow release of the peptide copper complex may be provided from the high molecular weight pegylated peptide copper complex.

Aqueous solutions of the disclosed PEG-peptide copper complexes may be prepared by methods that are well known to one skilled in the art. For example, an amount of a suitable peptide is coupled to a suitable PEG to form the PEG-peptide. The PEG-peptide is dissolved in water followed by the addition of a copper salt in the desired molar ratio to yield the desired solution of the PEG- peptide copper complex. Examples of copper salts that may be used are cupric chloride and cupric acetate. When aqueous solutions of peptide copper complexes are prepared, the solutions are neutralized, typically with NaOH.

In view of the previously noted beneficial health and cosmetic applications of compositions comprising peptide copper complexes, the compositions of the present invention, in certain embodiments, may be formulated for use as pharmaceutical or cosmetic products. Accordingly, the composition of the present invention, in certain embodiments, may further comprise an inert and physiologically-acceptable carrier or diluent, where, in related, more specific embodiments, the carrier or diluent is water, physiological saline, bacteriostatic saline, a pharmaceutically or cosmetically acceptable gel or cream, a short chain alcoholic solution, or a short chain glycol.

Further, the compositions disclosed herein may comprise, in addition to PEG-peptide copper complex, an active agent. The expression “active agent,” as used herein, refers to a compound or substance that provides benefits to the skin and/or provides desirable properties to a composition formulated as a cosmetic preparation. Active agents include, as examples, active drug substances, active cosmetic substances, sunscreen agents, skin lightening agents, skin conditioning agents, skin protectants, emollients and humectants. In one embodiment, the active agent is an active drug substance. The expression, “active drug substance,” as used herein, refers to a chemical or biological moiety that has been shown to alter either the composition or function of the body.

In yet another embodiment, the active agent is an active cosmetic substance. The expression, “active cosmetic substance,” as used herein, refers to compounds, mixtures, or extracts that have various positive effects on the skin of a patient. In related, more particular embodiments, the active cosmetic substance is, in one such embodiment, retinol, retinoic acid, or a derivative thereof; and, in another such embodiment, the active cosmetic substance is allantoin, tocopherol, tocopherol derivatives, niacinamide, phytosterols, isoflavones, panthenol, panthenol derivatives, bisabolol or farnesol.

In a further related and more particular embodiment, the active cosmetic substance is a phytochemical compound. As is well understood by one of ordinary skill in the art, a phytochemical compound may either be in a purified form or as present in extracts derived from various plants. Examples of phytochemical cosmetically-active substances include, but are not limited to, any of the anti-oxidant pigments that are naturally present in, and impart color to, fruits and vegetables, as well present in the roots, bark, leaves, flowers and seeds of plants. Polyphenols and carotenoids are examples of phytochemical compounds. Flavanoids, flavonoids and their derivatives, flavolignans and polyphenolic rhizomes, represent some of the more significant polyphenols, with regard to having potent anti-oxidant and anti-inflammatory properties. Examples of plant extracts that provide such active cosmetic substances are extracts of the genus Camellia, including Camellia sinensis (i.e., green tea) and Camellia assaimic, licorice, sea whip, aloe vera, chamomile, and the like.

In another embodiment of the disclosed composition comprising an active agent, the active agent is a skin lightening agent, a sunscreen agent, a skin conditioning agent, a skin protectant, an emollient, a humectant, or a mixture thereof. Suitable sunscreen agents absorb, reflect, or scatter radiation in the UV range at wavelengths ranging from 290 to 400 nanometers. Specific examples include, but are not limited to, benzophenone-3 (oxybenzone), butyl methoxydibenzoylmethane (Avobenzone), ethylhexyl methoxycinnamate (octyl methoxycinnamate), ethylhexyl salicylate (octyl salicylate), homosalate, PABA (aminobenzoic acid), titanium dioxide, and zinc oxide. One skilled in the art will appreciate that other sunscreen agents may be included in the compositions of the present invention.

Suitable skin lightening agents include, but are not limited to, ascorbic acid and derivatives thereof; kojic acid and derivatives thereof; hydroquinone; azelaic acid; and various plant extracts, such as those from licorice, grape seed, and bear berry. Those skilled in the art will appreciate that other skin lightening agents may be included in the compositions of the present invention.

Suitable skin conditioning agents typically comprise a substance that enhances the appearance of dry or damaged skin, as well as a material that adheres to the skin to reduce flaking, restore suppleness, and generally improve the appearance of skin. Representative examples of a skin conditioning agent that may be used include: acetyl cysteine, adenosine, algae extract, allantoin and derivatives, aloe barbadensis extracts, butylene glycol, cycloethoxymethicone, dimethicone copolyols, gelatin, glycosaminoglycans, glycosphingolipids, malt extract, maltodextrin, phytosterols, stearamidopropyl betaine, and stearyl palmitate. A skin conditioning agent, other than those listed above, may also be used, as is readily appreciated by those skilled in the art.

Suitable skin protectants, defined herein as a compound that protects injured or exposed skin or mucous membrane surfaces from harmful or irritating external compounds, include: algae extract, allantoin, cerebrosides, dimethicone, mineral oil, petrolatum, potassium gluconate, and talc. Those skilled in the art will readily appreciate that a skin protectant, other than those listed above, may be included in the compositions.

An emollient, as the term is used herein, is a cosmetic ingredient that can help skin maintain a soft, smooth, and pliable appearance. Emollients are able to provide these benefits, largely owing to their ability to remain on the skin surface, or in the stratum corneum, to act as a lubricant and reduce flaking. Some examples of an emollient, suitable for use in the above-disclosed, specific embodiment of this invention, are: acetylated lanolin, butoxyethyl stearate, C₁₈-C₃₆ acid glycol ester, C₁₂-C₁₃ alkyl lactate, dimyristyl tartrate, disteareth-5 lauroyl glutamate, isotridecyl isononanoate, raffinose, stearyl citrate, sunflower seed oil glycerides, and tocopheryl glucoside. Those skilled in the art will readily appreciate that emollients, other than those listed above, may also be used.

Humectants are cosmetic ingredients that help maintain moisture levels in skin. Some examples of suitable humectants are: aloe barbadensis leaf extract, 2,3-butanediol, erythritol, fructose, glucose, glycerin, honey, hydrolyzed wheat protein, inositol, sorbitol, sucrose, and urea. Other humectants may be used for yet additional embodiments of this invention, as will be appreciated by those skilled in the art.

In a further embodiment of the composition of the present invention, the composition comprises a fatty alcohol, a fatty acid, an organic base, an inorganic base, a preserving agent, a wax ester, a steroid alcohol, a triglyceride ester, a phospholipid, a polyhydric alcohol ester, a fatty alcohol ether, a hydrophilic lanolin derivative, a hydrophilic beeswax derivative, a cocoa butter wax, a silicon oil, a pH balancer, a cellulose derivative, a hydrocarbon oil, or a mixture thereof. Non-limiting examples of a suitable phospholipid include lecithin and cephalin. Suitable hydrocarbon oils include, but are not limited to, palm oil, coconut oil, and mineral oil.

Additional ingredients may be included in the above compositions to vary the texture, viscosity, color and/or appearance thereof, as is appreciated by one of ordinary skill in the art. Accordingly, in a further embodiment, the present invention is directed to a composition that comprises, in addition to the PEG-peptide copper complex, an emulsifying agent, a surfactant, and/or a thickening agent.

As a specific example, an emulsifier and a surfactant may be included in a composition that is formulated as an emulsion. Either a water-in-oil or oil-in-water emulsion may be formulated. Examples of suitable surfactants and emulsifying agents include: nonionic ethoxylated and nonethoxylated surfactants, C₁₈-C₃₆ acid glycol ester, C₉-C₁₅ alkyl phosphate, dextrin laurate, lecithin, lysolecithin, polyethylene glycol stearamine, sodium caprylate, and sodium cocoate. Other surfactants and emulsifiers may be used, as will be appreciated by one of ordinary skill in the art.

Examples of a thickening (i.e., viscosity increasing) agent that is suitable for inclusion in the composition include, but are not limited to, those agents commonly used in skin care preparations. More specifically, such examples include acrylamides, agarose, amylopectin, calcium alginate, calcium carboxymethyl cellulose, carbomer, cellulose gum, gelatin, hydrogenated tallow, hydroxyethylcellulose, hydroxypropyl methylcellulose, pectin, various polyethylene glycols, polyacrylic acid, polymethacrylic acid, polyvinyl alcohol, sodium carrageenan, xanthan gum, and yeast beta-glucan. Thickening agents other than those listed above may also be used in related embodiments of the present invention.

The compositions of the present invention may be applied topically to human skin. Accordingly, such a composition may be formulated, in a further embodiment, as a liquid, cream, gel, oil, fluid cream or milk, lotion, emulsion, or microemulsion. In a related embodiment, the composition further comprises an excipient adapted for application to the face and neck. Such an excipient should have a high affinity for the skin, be well tolerated, stable, and yield a consistency that allows for easy and pleasant utilization.

The present invention is also directed to medical devices that comprise a disclosed PEG-peptide copper complex. Non-limiting examples of such a device are implantable medical devices and wound dressings, such as sterile gauze pads, impregnated with a composition of the present invention in the form of a gel or solution for application to a wound.

The present invention is also directed to the combination of a PEG-peptide copper complex formulated in combination with a soft or hard tissue filler for use in, for example, correction of dermal or hard tissue defects, fine lines and wrinkles resulting from photoaging, facial wasting associated with HAART (Highly Active Anti-Retroviral Treatment) therapy of HIV infections, and the like. Such a composition may be formulated to be suitable for subcutaneous injection. As noted previously, such pegylated peptide copper complexes increase the residency time of the peptide copper complex in the soft or hard tissue filler material allowing additional time for the desired biological action to occur. Suitable soft tissue fillers include hyaluronic acid, collagen, polylactic acid, polyglycolic acid, polyacrylic acid, silicon fluid and the like. Suitable hard tissue fillers include collagen, polylactic acid, polyglycolic acid, glass beads, polyacrylic acid, and the like.

The following examples are provided for the purpose of illustration, not limitation.

### EXAMPLES

The examples that follow illustrate the preparation, characterization and utility of certain compositions of the present invention.

### Example 1

This example describes the coupling of a representative peptide, glycyl-L-histidyl-L-lysine to a low molecular weight PEG molecule, followed by complexation of the PEG-peptide with copper(II).

A solution of a PEG succinimidyl propionate derivative having a molecular weight of about 5,000 daltons is reacted with 2 molar equivalents of glycyl-L-histidyl-L-lysine (GHK) at ambient temperature. The pH is then adjusted to about 9.0 with sodium carbonate. After 30 minutes, the reaction is stopped by lowering the pH to 5.0 with HCl.

The extent of reaction can be determined by measuring the remaining GHK by HPLC relative to the amount in the starting material.

The copper complex is formed by adding an equal volume of an aqueous solution containing 1 molar equivalent of copper(II) chloride to the solution and adjusting the pH to 6.5-7.0. The resulting deep blue color of the solution is indicative of the formation of the PEG-peptide copper complex.

### Example 2

This example describes the coupling of a representative peptide, glycyl-L-histidyl-L-lysine to a high molecular weight PEG molecule, followed by complexation of the PEG-peptide with copper(II).

A solution of a PCE succinimidyl propionate derivative having a molecular weight of about 30,000 daltons is reacted with 2 molar equivalents of glycyl-L-histidyl-L-lysine (GHK) at ambient temperature. The pH is then adjusted to about 9.0 with sodium carbonate. After 30 minutes, the reaction is stopped by lowering the pH to 5.0 with HCl.

The extent of reaction can be determined by measuring the remaining GHK by HPLC relative to the amount in the starting material.

The copper complex is formed by adding an equal volume of an aqueous solution containing 1 molar equivalent of copper(II) chloride to the solution and adjusting the pH to 6.5-7.0. The resulting deep blue color of the solution is indicative of the formation of the PEG-peptide copper complex.

### Example 3

This example describes the coupling of a representative peptide, L-alanyl-L-histidyl-L-lysine to a high molecular weight branched PEG molecule followed by complexation of the peptide with copper(II).

A solution of a branched methyl-PEG aldehyde derivative having a molecular weight of about 30,000 daltons is reacted with 2 molar equivalents of L- alanyl-L-histidyl-L-lysine (AHK) at ambient temperature. The pH is then adjusted to about 9.0 with sodium carbonate. After 30 minutes, the reaction is stopped by lowering the pH to 5.0 with HCl.

The extent of reaction is determined by measuring the remaining AHK by HPLC relative to the amount in the starting material.

The copper complex is formed by adding an equal volume of an aqueous solution containing 1 molar equivalent of copper(II) chloride to the solution and adjusting the pH to 5.5-6.0. The resulting deep blue color of the solution is indicative of the formation of the PEG-peptide copper complex.

### Example 4

This example describes the formulation of a PEG-peptide copper complex composition of the present invention in a pharmaceutical composition suitable for intradermal injection.

A solution of a PEG-peptide copper complex is prepared at a concentration of 0.1% by weight according to the foregoing examples and is adjusted with sodium chloride to be isotonic. The resulting isotonic solution is filtered though a sterile 0.2 micron filter into a sterile sealed vial.

The resulting solution is suitable for intradermal injection.

### Example 5

This example describes the formulation of a PEG-peptide copper complex composition of the present invention in a pharmaceutical composition comprising a soft tissue filler, suitable for intradermal injection.

A solution of a PEG-peptide copper complex is prepared at a concentration of 0.2% by weight according to the foregoing examples and is adjusted with sodium chloride to be isotonic. The resulting isotonic solution is filtered though a sterile 0.2 micron filter into a sterile sealed vial. This solution is then mixed in equal volume with a commercially available soft tissue filler, Restyline, which is composed of an isotonic suspension of modified hyaluronic acid.

The resulting mixed suspension of PEG-peptide copper complex and hyaluronic acid is suitable for intradermal injection as a soft tissue filler for the treatment of fine lines and wrinkles.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A composition comprising a peptide copper complex coupled to a polyethylene glycol molecule.

2. The composition of claim 1 wherein the peptide copper complex has the formula:
[R₁—R₂—R₃]:copper(II)
wherein R₁ is an amino acid or an amino acid derivative, R₂ is an amino acid or an amino acid derivative, and R₃ is at least one amino acid or amino acid derivative or a chemical moiety.

3. The composition of claim 2 wherein R₂ is L-histidyl.

4. The composition of claim 3 wherein R₁ and R₃ are both naturally occurring amino acids.

5. The composition of claim 3 wherein R₁ is a naturally occurring amino acid and R₃ is L-lysine.

6. The composition of claim 3 wherein R₁ is glycyl and R₃ is L-lysine.

7. The composition of claim 3 wherein R₁ is L-alanyl and R₃ is L-lysine.

8. The composition of claim 2 wherein R₂ is L-histidyl(R), wherein R is 3-methyl, 5 methyl, 3-ethyl or 5-ethyl.

9. The composition of claim 8 wherein R₁ and R₃ are both naturally occurring amino acids.

10. The composition of claim 2 wherein R₂ is a L-arginyl.

11. The composition of claim 10 wherein R₁ and R₃ are both naturally occurring amino acids.

12. The composition of claim 1 wherein the polyethylene glycol molecule has a molecular weight of about 5,000 daltons.

13. The composition of claim 1 wherein the polyethylene glycol molecule has a molecular weight of about 20,000 daltons.

14. The composition of claim 1 wherein the polyethylene glycol molecule has a molecular weight of about 30,000 daltons.

15. The composition of claim 1 wherein the polyethylene glycol molecule has a molecular weight of about 40,000 daltons.

16. The composition of claim 1 wherein the polyethylene glycol molecule is a linear polymer.

17. The composition of claim 1 wherein the polyethylene glycol molecule is a branched polymer.

18. A cosmetic composition comprising the composition of claim 1.

19. The cosmetic composition of claim 18, further comprising an active agent selected from the group consisting of active drug substances, active cosmetic substances, sunscreen agents, skin lightening agents, skin conditioning agents, skin protectants, emollients and humectants.

20. The cosmetic composition of claim 18, further comprising a fatty alcohol, a fatty acid, an organic base, an inorganic base, a preserving agent, a wax ester, a steroid alcohol, a triglyceride ester, a phospholipid, a polyhydric alcohol ester, a fatty alcohol ether, a hydrophilic lanolin derivative, a hydrophilic beeswax derivative, a cocoa butter wax, a silicon oil, a pH balancer, a cellulose derivative, a hydrocarbon oil, or a mixture thereof.

21. The cosmetic composition of claim 18, further comprising an emulsifying agent, a surfactant, a thickening agent, or a mixture thereof.

22. The cosmetic composition of claim 18, further comprising a soft tissue filler.

23. The cosmetic composition of claim 22 wherein the soft tissue filler is selected from the group consisting of hyaluronic acid, collagen, polylactic acid, polyglycolic acid, polyacrylic acid, and silicon fluid.

24. The cosmetic composition of claim 18, further comprising a hard tissue filler.

25. The cosmetic composition of claim 24 wherein the hard tissue filler is selected from the group consisting of collagen, polylactic acid, polyglycolic acid, glass beads and polyactylic acid.

26. A pharmaceutical composition comprising the composition of claim 1 and an inert and physiologically-acceptable carrier or diluent.

27. The pharmaceutical composition of claim 26 wherein the pharmaceutical composition is suitable for intradermal injection.

28. The pharmaceutical composition of claim 26 wherein the pharmaceutical composition is a topical formulation suitable for wound care.

29. The pharmaceutical composition of claim 26, further comprising a soft tissue filler.

30. The pharmaceutical composition of claim 29 wherein the soft tissue filler is selected from the group consisting of hyaluronic acid, collagen, polylactic acid, polyglycolic acid, polyacrylic acid, and silicon fluid.

31. The pharmaceutical composition of claim 26, further comprising a hard tissue filler.

32. The pharmaceutical composition of claim 31 wherein the hard tissue filler is selected from the group consisting of collagen, polylactic acid, polyglycolic acid, glass beads and polyactylic acid.

33. A medical device comprising the composition of claim 1.

34. The medical device of claim 33 wherein the medical device is a wound dressing.

35. The medical device of claim 33 wherein the medical device is an implantable medical device.
